(19) United States
(12) Patent Application Publication
(10) Pub. No.: US 2026/0198983 A1
(43) Pub. Date: Jul. 16, 2026

(54) COMPLIANT ORTHOPEDIC DRIVER

(71) Applicant: GLOBUS MEDICAL, INC., AUDUBON, PA (US)

(72) Inventors: Olivier Chappuis, Lutry (CH); Szymon Kostrzewski, Lausanne (CH)

(21) Appl. No.: 19/564,730

(22) Filed: Mar. 12, 2026

Related U.S. Application Data
(63) Continuation of application No. 18/909,070, filed on Oct. 8, 2024, now Pat. No. 12,575,869.
Application No. 18/909,070 is a continuation of application No. 18/528,855, filed on Dec. 5, 2023, now Pat. No. 12,121,278.
Application No. 18/528,855 is a continuation of application No. 17/726,696, filed on Apr. 22, 2022, now Pat. No. 11,832,863.
Application No. 17/726,696 is a continuation of application No. 16/180,381, filed on Nov. 5, 2018, now Pat. No. 11,337,742.

(51) Int. Cl. A61B 17/88; A61B 34/30; A61B 17/00; A61B 17/90; A61B 34/20

(52) U.S. Cl. CPC A61B 17/8875; A61B 34/30; A61B 17/90; A61B 2017/00862; A61B 2034/2072

(57) ABSTRACT

Embodiments of a compliant orthopedic driver are disclosed herein. In some embodiments, compliant orthopedic driver includes a body extending from a proximal end to a distal end along a driver axis; a driver tip disposed at the distal end of the body, wherein the body includes at least one compliant portion configured to allow the driver flex about at least two axes transverse to the driver axis.

### CROSS REFERENCE TO RELATED APPLICATIONS

This patent application is a continuation of U.S. Patent Application Serial No. 18/909,070 filed on October 8, 2024, which is a continuation of U.S. Patent Application No. 18/528,855 filed on December 5, 2023, which is a continuation of Patent Application No. 17/726,696 filed on April 22, 2022, which is a continuation of Patent Application No. 16/180,381 filed on November 5, 2018, all of which are incorporated in their entirety herein.

### FIELD OF THE INVENTION

The invention generally relates to devices and methods that improve surgical procedures by, for example, providing a working space for the procedure and improving the surgical conditions for the practitioner of a procedure.

### BACKGROUND OF THE INVENTION

During spinal, orthopedic and general surgeries, screws are often used to fix implants and other mechanical constructs to bony structure. There are an increasing number of robotic systems and various types of instrument guides available on the market. The goal of such apparatuses is to assist surgeons in drilling, tapping and/or screw placement along a desired trajectory. The usage of guides and robotic systems to maintain the desired trajectory can lead to undesirable locking between the driver (e.g., a screwdriver) and the fixation element (e.g., a screw) in a manner which makes it difficult to decouple the driver from the fixation element. This difficult can negatively impact the surgical procedure and possibly the final result of the procedure.

For example, in the case of pedicle screw placement along a spine, a guide (robotic or handheld) is first used to guide a drill in the direction of a desired trajectory. Subsequently, the drill is inserted through the guide to begin drilling the hole in a vertebra. However, upon contact with the bone, contact forces (action and reaction) may cause the less rigid structure (typically the bone/vertebra) to move away from its initial position. If the trajectory of the drill is not corrected to compensate for these contact forces, the actual drilled trajectory will not be aligned with the desired trajectory. After the hole is drilled, other hole preparation tasks may be performed (e.g., tapping, bone breach testing, etc.). Finally, the implant is placed in the bone. Since implants do not typically have cutting abilities, the implant (e.g., a screw) follows the drilled hole trajectory. Because the driver being used to install the implant extends through the guide, which extends along the desired trajectory, and the implant extends along the drilled hole trajectory, which is not collinear with the desired trajectory, there will be action-reaction forces and torques at the interface of the implant and the driver. This interface is typically configured to have minimal mechanical backlash and a rigid connection (e.g., torx interface, hex interface, etc.). However, this interface does not adapt well to off-axis use and, as such, will result in the locking of the driver in the implant. The principal, underlying cause of this locking is over-constraint in the driver. When the driver is locked in the implant and cannot be decoupled therefrom, typical troubleshooting techniques are moving the guide, which may delay the surgery and/or result in improper implantation of the implant.

Therefore, a need exists for an orthopedic driver that overcomes or minimizes these and other problems.

### SUMMARY

Embodiments of a compliant orthopedic driver are disclosed herein. In some embodiments, compliant orthopedic driver includes a body extending from a proximal end to a distal end along a driver axis; a driver tip disposed at the distal end of the body, wherein the body includes at least one compliant portion configured to allow the driver flex about at least two axes transverse to the driver axis.

Embodiments of a compliant orthopedic driver are disclosed herein. In some embodiments, compliant orthopedic driver includes a body extending from a proximal end to a distal end along a driver axis; a driver tip disposed at the distal end of the body, wherein the body includes at least one compliant portion configured to allow the driver flex about at least two axes transverse to the driver axis wherein the body includes a first compliant portion and a second compliant portion spaced apart from the first compliant portion, and wherein each of the first and second compliant portions are configured to allow the driver flex about at least two axes transverse to the driver axis.

### BRIEF DESCRIPTION OF THE DRAWINGS

The invention will be more readily understood with reference to the embodiments thereof illustrated in the attached figures, in which:

FIG. 1A is an isometric view of a compliant driver in accordance with an embodiment of the present disclosure;

FIG. 1B is a front view of the compliant drive of FIG. 1A;

FIG. 1C is a top view of the compliant driver of FIG. 1A;

FIG. 1D is an isometric view of a compliant driver in accordance with an embodiment of the present disclosure;

FIG. 1E is a front view of the compliant drive of FIG. 1D;

FIG. 2A is an isometric view of a compliant driver in accordance with another embodiment of the present disclosure;

FIG. 2B is a front view of the compliant drive of FIG. 2A;

FIG. 2C is a top view of the compliant driver of FIG. 2A;

FIG. 3A is an isometric view of a compliant driver in accordance with another embodiment of the present disclosure;

FIG. 3B is a front view of the compliant driver of FIG. 3A;

FIG. 4A is an isometric view of a compliant driver in accordance with another embodiment of the present disclosure;

FIG. 4B is a front view of the compliant driver of FIG. 4A; and

FIGS. 5A-5B depict an exemplary robotic arm with which a compliant driver according to embodiments of the present disclosure may be used.

### DETAILED DESCRIPTION

Embodiments of the invention will now be described. The following detailed description of the invention is not intended to be illustrative of all embodiments. In describing embodiments of the present invention, specific terminology is employed for the sake of clarity. However, the invention is not intended to be limited to the specific terminology so selected. It is to be understood that each specific element includes all technical equivalents that operate in a similar manner to accomplish a similar purpose.

Typically, an orthopedic driver (e.g., a screwdriver) requires the two degrees of freedom (DOFs) to be locked in order to fulfil its main function (i.e., insert a screw): (1) Rotation around the driver axis and (2) translation along the driver axis. This means that when an implant is attached to the driver tip, the implant should not be able to rotate around nor translate along the driver axis. When the four remaining DOFs between driver and implant are also locked, a mechanical over-constrain results at the implant-driver interface due to non-collinearity of a trajectory of a hole drilled into a bone and instrument guiding trajectory. This over-constrain is the root cause of screw locking issues.

As such, it is necessary to add compliance to the driver to allow the driver tip to adapt or flex to the screw trajectory and, therefore, eliminate the mechanical over-constrain at the implant-driver interface. One way to add compliance to the driver is to incorporate a kinematic chain into the driver. In some embodiments, the kinematic chain may include two or more universal joints in series, each of which adds two DOFs (rotation about a first axis and rotation about a second axis) for a total of four DOFs. In other embodiments, the kinematic chain may include a universal joint (two DOFs, as explained above) and a semi-ball joint (three DOFs – rotation about three axes).

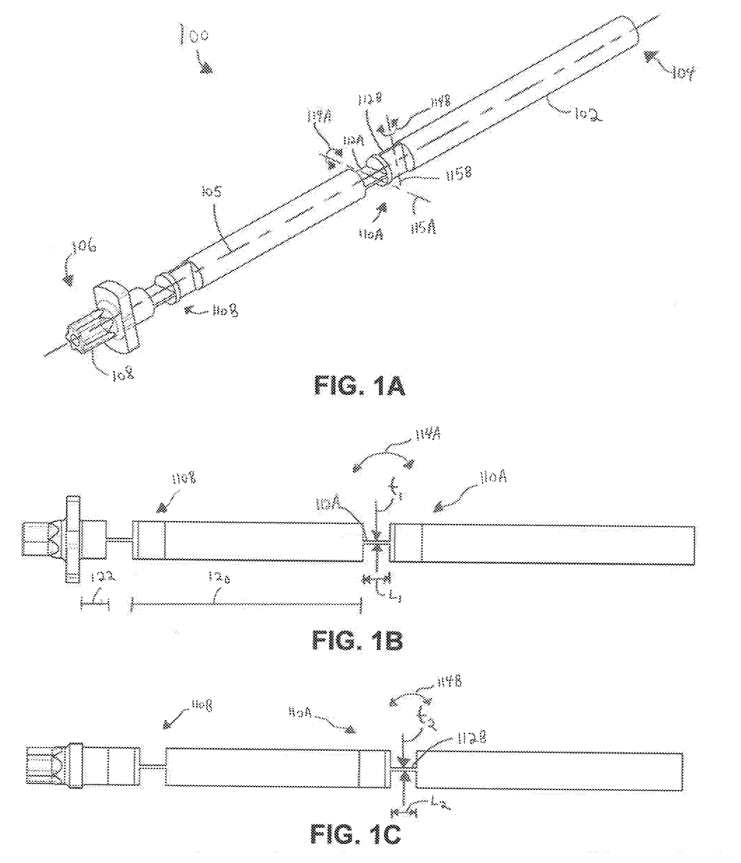

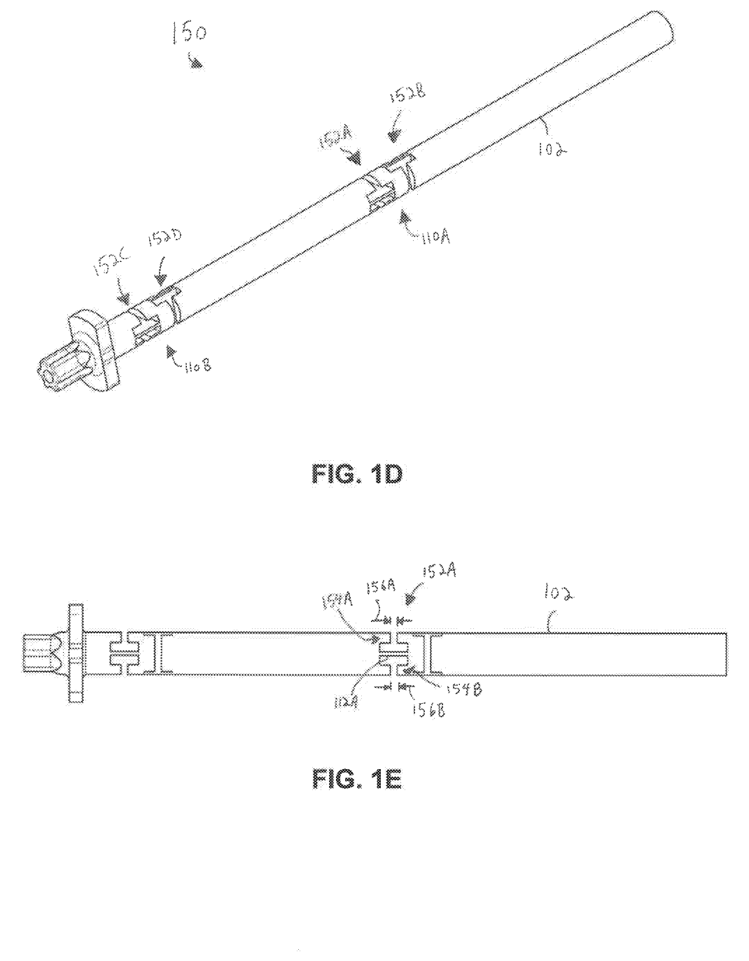

FIGS. 1A-1C depict a compliant orthopedic driver 100 (e.g., a screwdriver) in accordance with embodiments of the present disclosure. In some embodiments, the driver 100 includes a body 102 extending from a proximal end 104 to a distal end 106 along a driver axis 105. The body 102 terminates at a driver tip 108 at the distal end 106. The driver tip 108 may have any shape (e.g., torx, hex, cross, etc.) corresponding to the shape of a recess in the head of a screw (not shown) to be implanted using the driver 100. In the embodiment depicted in FIGS. 1A-1C, the two universal joints are implemented by two pairs of blade hinges 110A, 110B. The two pairs of blade hinges 110A, 110B are separated by a first distance 120. The second pair of blade hinges 110B is disposed a second distance 122 from the driver tip 108. The first and second distances 120, 122 depend on the surgical procedure (e.g., spine, trauma, etc.) and the tools being used (e.g., robotic guide, handheld guide, etc.). In this embodiment, the driver tip 108 exhibits 4 DOFs. The two locked DOFs are the rotation around and translation along the driver axis 105. In some embodiments, mechanical end-stops (not shown) may be incorporated to protect the hinges against over travel.

Because each pair of hinges is identical, the first pair of blade hinges 110A will be described for brevity. The first pair of blade hinges 110A includes a first blade 112A and a second blade 112B which extends transversely to the first blade 112A. In some embodiments, the second blade 112B is perpendicular to the first blade 112A. The first blade 112A has a first length L₁ and a first thickness t₁ and the second blade has a second length L₂ and a second thickness t₂. In some embodiments, the first length L₁ is equivalent to the second length L₂. In some embodiment, the first thickness t₁ is equivalent to the second thickness t₂. The first length L₁ and the first thickness t₁ are configured to allow the driver 100 to flex about a first axis 115A as indicated by arrow 114A. Similarly, the second length L₂ and the second thickness t₂ are configured to allow the driver 100 to flex about a second axis 115B as indicated by arrow 114B. In some embodiments, the first and second axes 115A, 115B are perpendicular to the driver axis 105. In some embodiments, first and second blades 112A, 112B are configured to allow for a degree of flexure between 0° and 10°. In some embodiments, the first and second blades 112A, 112B are adjacent to one another, as depicted in FIGS. 1A-1C. In some embodiments, the first and second blades 112A, 112B may alternatively be spaced apart by a predetermined distance. In some embodiments, all sharp edges/corners of the blades may be rounded to reduce high stress concentrations.

FIGS. 1D and 1E depict a compliant orthopedic driver 150 in accordance with another embodiment of the present disclosure. The driver includes all of the elements discussed above with respect to the driver 100. As such, a description of those features will be omitted here for clarity. The orthopedic driver 150 additionally includes first and second pairs of stops 152A, 152B corresponding to the first pair of blade hinges 110A and third and fourth pairs of stops 152C, 152D corresponding to the second pair of blade hinges 110B. As depicted in FIGS. 1D and 1E, each pair of stops is disposed on opposite sides of the blades to prevent over travel (i.e., limit the amount of travel) of the driver 150 in either direction of flexure. Because all four pairs of stops are substantially similar, only the first pair of stops 152A will be described. It should be understood that this description also applies to the other pairs of stops. As shown more clearly in FIG. 1E, the first pair of stops 152A includes a first pair of cantilevered portions 154A disposed on a first side of the first blade 112A and a second pair of cantilevered portions 154B disposed on a second side opposite the first side of the first blade 112A. The first and second pairs of cantilevered portions 154A, 154B extend from the body 102 over the first blade 112A. The first pair of cantilevered portions 154A are separated by a first predetermined distance 156A. The second pair of cantilevered portions 154B are separated by a second predetermined distance 156B. In some embodiments, the first and second predetermined distances 156A, 156B are equivalent; such that the amount flexure in both directions is equally limited. In some embodiments, the first and second predetermined distances 156A, 156B are not equivalent; such that flexure in one direction is limited more than flexure in the opposite direction.

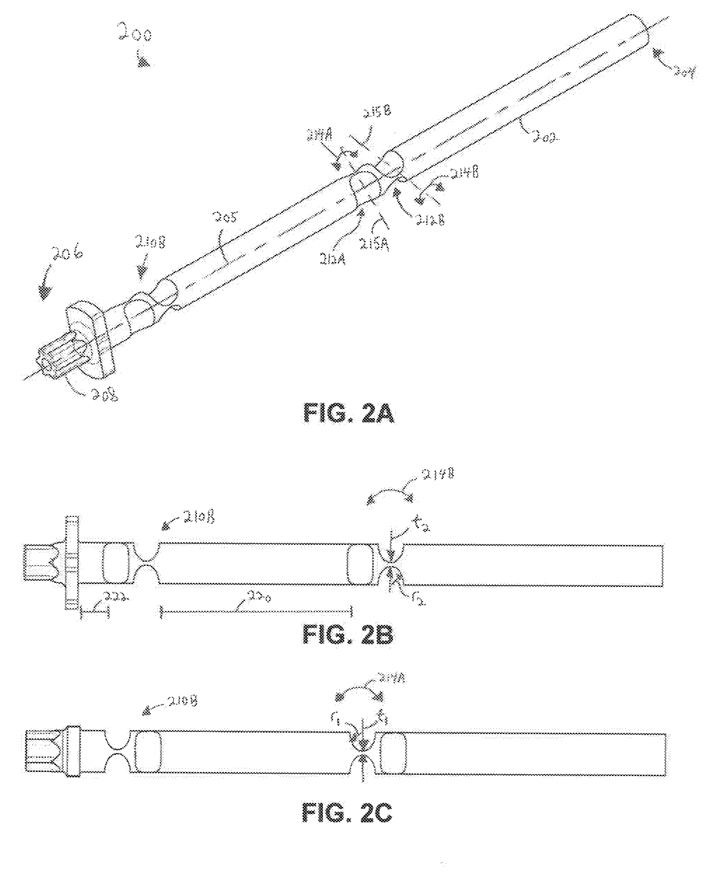

FIGS. 2A-2C depict a compliant orthopedic driver 200 (e.g., a screwdriver) in accordance with another embodiment of the present disclosure. The driver 200 functions similarly to the drive 100 described above. In some embodiments, the driver 200 includes a body 202 extending from a proximal end 204 to a distal end 206 along a driver axis 205. The body 202 terminates at a driver tip 208 at the distal end 206. The driver tip 208 may have any shape (e.g., torx, hex, cross, etc.) corresponding to the shape of a recess in the head of a screw (not shown) to be implanted using the driver 200. In the embodiment depicted in FIGS. 2A-2C, the two universal joints are implemented by two pairs of notch hinges 210A, 210B. The two pairs of notch hinges 210A, 210B are separated by a first distance 220. The second pair of notch hinges 210B is disposed a second distance 222 from the driver tip 208. The first and second distances 220, 222 depend on the surgical procedure (e.g., spine, trauma, etc.) and the tools being used (e.g., robotic guide, handheld guide, etc.). In this embodiment, the driver tip 208 also exhibits 4 DOFs. The two locked DOFs are again the rotation around and translation along the driver axis 205. In some embodiments, mechanical end-stops (not shown) may be incorporated to protect the hinges against over travel.

Because each pair of hinges is identical, the first pair of notch hinges 210A will be described for brevity. The first pair of notch hinges 210A includes a first pair of notches 212A and a second pair of notches 212B which extends transversely to the first blade 212A. In some embodiments, the second pair of notches 212B are angularly offset with respect to the first pair of notches 212A by 90°. Each of the first pair of notches 212A has a first radius r₁. A first thickness t₁ separates the first pair of notches 212A (FIG. 2C). Each of the second pair of notches 210B has a second radius r₂. A second thickness t₂ separates the second pair of notches 212B (FIG. 2C). In some embodiments, the first radius r₁ is equivalent to the second radius r₂. In some embodiments, the first thickness t₁ is equivalent to the second thickness t₂. The first radius r₁ and the first thickness t₁ are configured to allow the driver 200 to flex about a first axis 215A as indicated by arrow 214A. Similarly, the second radius r₂ and the second thickness t₂ are configured to allow the driver 200 to flex about a second axis 215B as indicated by arrow 214B. In some embodiments, the first and second axes 215A, 215B are perpendicular to the driver axis 205. In some embodiments, first and second pairs of notches 212A, 212B are configured to allow for a degree of flexure between 0° and 10°. In some embodiments, the first and second pairs of notches 212A, 212B are adjacent to one another, as depicted in FIGS. 2A-2C. In some embodiments, the first and second pairs of notches 212A, 212B may alternatively be spaced apart by a predetermined distance. The notches 212A, 212B advantageously allow for gradual flexure without high stress areas associated with sharp corners. In some embodiments, the driver 200 may also include stops as described above with respect to FIGS. 1D and 1E to limit the amount of flexure of the driver.

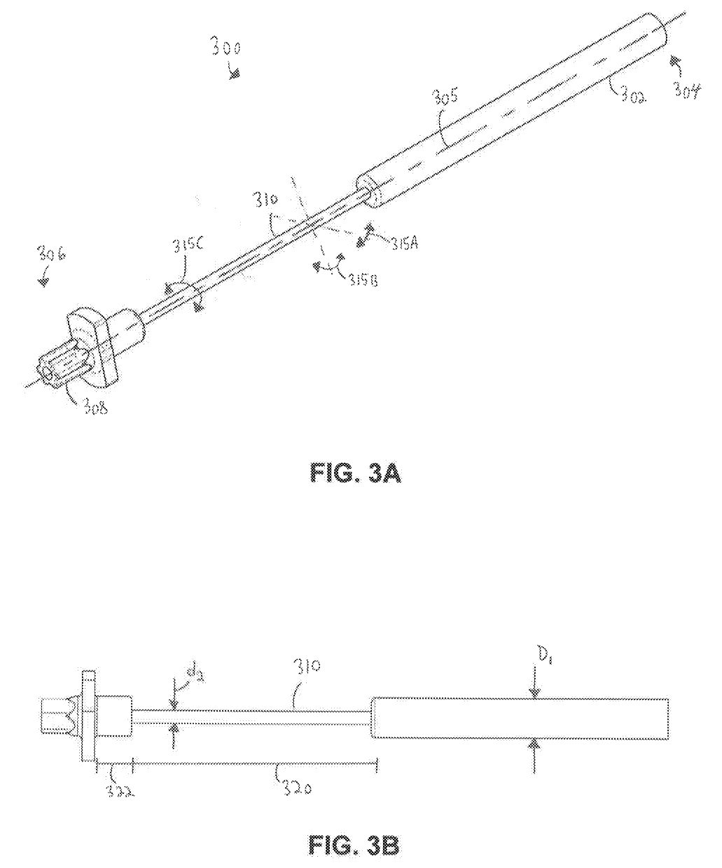

FIGS. 3A and 3B depict a compliant orthopedic driver 300 (e.g., a screwdriver) in accordance with another embodiment of the present disclosure. The driver 300 functions somewhat similarly to the drivers 100, 200 described above. In some embodiments, the driver 300 includes a body 302 extending from a proximal end 304 to a distal end 306 along a driver axis 305. The body 302 terminates at a driver tip 308 at the distal end 306. The driver tip 308 may have any shape (e.g., torx, hex, cross, etc.) corresponding to the shape of a recess in the head of a screw (not shown) to be implanted using the driver 300. The driver 300 incorporates the semi-ball joint method, which is implemented by having a reduced diameter section 310. The reduced diameter section provides 5 DOFs: flexure about a first axis 314A, slight translation along the first axis 314A, flexure about a second axis 314B, slight translation along the second axis 314B, and torsion about the driver axis 305.

The reduced diameter section 310 has a first length 320 and is disposed a first distance 322 from the driver tip 308. The first length 320 and the first distance 322 depend on the surgical procedure (e.g., spine, trauma, etc.) and the tools being used (e.g., robotic guide, handheld guide, etc.). The reduced diameter section 310 has a second diameter d₂, which is smaller than a first diameter d₁ of the body 302. The first length 320 and the second diameter d2 dictate the amount of flexure of the driver 300. In some embodiments, the first length 320 and the diameter of the reduced diameter section 310 may be configured to allow for slight torsion about the driver axis 305. As such, the only fully locked DOF is the translation along the driver axis 305. The first length 320 is configured to allow for more torsion stiffness than flexure stiffness. In some embodiments, the driver 300 may also include stops as described above with respect to FIGS. 1D and 1E to limit the amount of flexure of the driver.

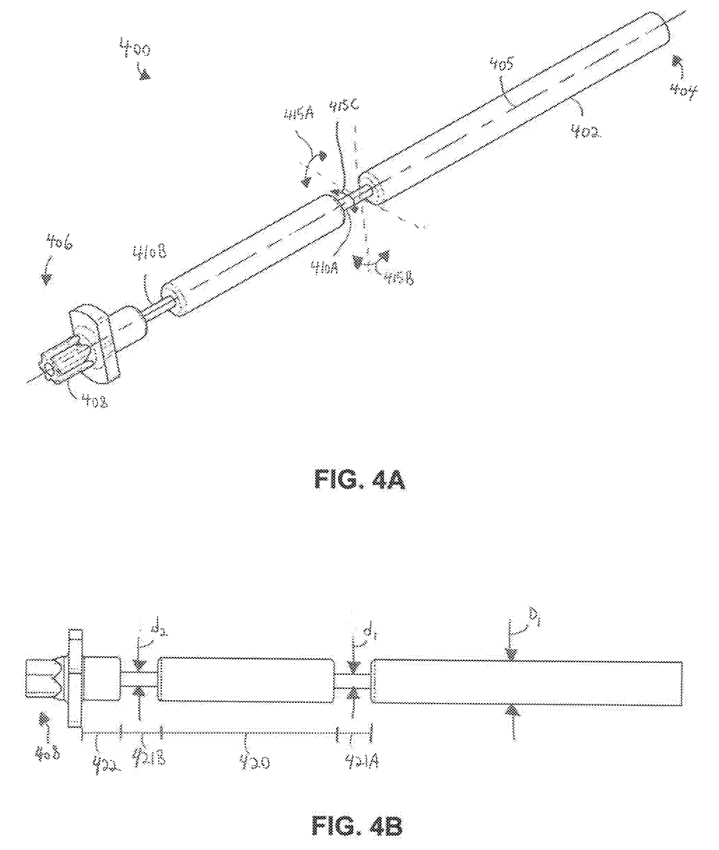

FIGS. 4A and 4B depict a compliant orthopedic driver 400 (e.g., a screwdriver) in accordance with another embodiment of the present disclosure. The driver 400 functions similarly to the driver 300 described above. In some embodiments, the driver 400 includes a body 402 extending from a proximal end 404 to a distal end 406 along a driver axis 405. The body 402 terminates at a driver tip 408 at the distal end 406. The driver tip 408 may have any shape (e.g., torx, hex, cross, etc.) corresponding to the shape of a recess in the head of a screw (not shown) to be implanted using the driver 400. The driver 400 also incorporates the semi-ball joint method, which is implemented by having a first reduced diameter section 410A and a second reduced diameter section 410B.

Because the second reduced diameter section 410B is identical to the first reduced diameter section, only the first reduced diameter section 410A will be described for brevity. The first reduced diameter section 410A has a first length 421A and a first reduced diameter d₁, which is less than a body diameter D₁. The first reduced diameter section 410A is disposed a first distance 420 from the second reduced diameter section 410B. The second reduced diameter section 410B is disposed a second distance 422 from the driver tip 408. The first and second lengths 421A, 421B, the first and second reduced diameters d₁, d₂, and the first and second distances 420, 422 all dicate the amount of flexure of the driver 400 depend on the surgical procedure (e.g., spine, trauma, etc.) and the tools being used (e.g., robotic guide, handheld guide, etc.). In some embodiments, these dimensions may be configured to allow for slight torsion about the driver axis 305. As such, the only fully locked DOF is the translation along the driver axis 405. Each of the first and second reduced diameter sections 410A, 410B provide the same DOFs as discussed above with respect to the reduced diameter section 310. However, having two shorter reduced diameter sections advantageously provides improved resistance to buckling under an axial load. In some embodiments, the driver 400 may also include stops as described above with respect to FIGS. 1D and 1E to limit the amount of flexure of the driver.

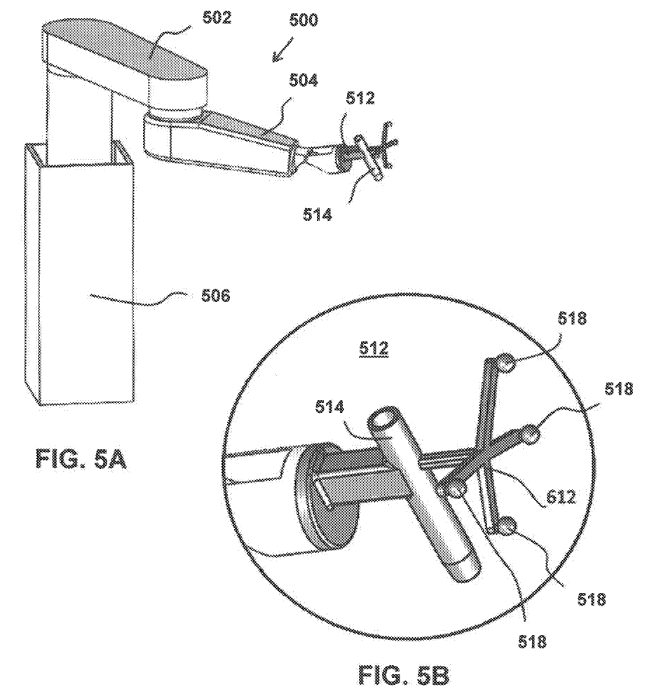

FIGS. 5A and 5B depict an exemplary robot arm with which a compliant driver (100, 200, 300, 400) in accordance with the present disclosure may be used. As noted above, the compliant driver (100, 200, 300, 400) may also be used with any other type of guide (e.g., handheld). FIG. 5A depicts part of a surgical robot system 500 with a robot 502 including base 506, robot arm 504, and end-effector 512. Other elements of the robot system, not illustrated, such as the display, cameras, etc. may also be present. FIG. 5B depicts a close-up view of the end-effector 512 with guide tube 514 and a plurality of tracking markers 518 rigidly affixed to the end-effector 512. In this embodiment, the plurality of tracking markers 118 are attached to the guide tube 112. When tracking an instrument such as, for example, the compliant driver (100, 200, 300, 400), the end-effector 512, or other object to be tracked in 3D, an array of tracking markers 518 may be rigidly attached to a portion of the instrument or end-effector 512. Preferably, the tracking markers 518 are attached such that the markers 518 are out of the way (e.g., not impeding the surgical operation, visibility, etc.). The markers 518 may be affixed to the instrument, end-effector 112, or other object to be tracked, for example, with an array 612.

Embodiments of the inventive driver advantageously solve the problem of driver-implant locking by preventing or substantially limiting such an occurrence by introducing adapted compliance in the driver. As a result, one exemplary realized benefit is the usability of such drivers in conjunction with robotic systems by decreasing the probability of occurrence of the implant locking effect to almost zero, without interfering with the general workflow or external functionality (proper implant function).

While the invention herein disclosed has been described with reference to specific embodiments and applications thereof, numerous modifications and variations can be made thereto by those skilled in the art without departing from the scope of the invention as set forth in the claims.

What is claimed is:

1. A surgical robotic system for driving a bone screw into bone comprising:
a surgical robot including a robot arm and an end effector, the end effector having a plurality of tracking markers and a guide tube;
a compliant orthopedic driver including:
a body extending from a proximal end to a distal end along a driver axis;
a driver tip disposed at the distal end of the body;
wherein the body includes at least one compliant portion including:
a first round notch adapted to flex about a first axis transverse to the driver axis; and
a second round notch longitudinally disposed from the first notch and adapted to flex about a second axis transverse to the driver axis, the first axis being angularly offset from the second axis; and
wherein the bone screw is driven into the bone using the compliant orthopedic driver,
wherein the compliant driver is positioned through the guide tube of the end effector.

2. The system of claim 1, wherein the first axis is angularly offset from the second axis by 90 degrees.

3. The system of claim 1, wherein the compliant portion includes a pair of oppositely disposed concave notches relative to the driver axis.

4. The system of claim 1, wherein the at least one compliant portion includes a pair of stops configured to limit an amount of flexure of the body about the at least two axes.

5. The system of claim 1, wherein the at least one compliant portion includes two compliant portions longitudinally spaced apart by a predetermined distance.

6. The system of claim 5, wherein the two compliant portions comprise a first pair of round notch hinges and a second pair of round notch hinges, each of which includes a first pair of round notches and a second pair of round notches angularly offset from the first pair of notches.

7. The system of claim 6, wherein the first pair of notches is perpendicular to the second pair of notches.

8. The system of claim 6, wherein each notch of the first pair of notches has a first predetermined radius and each notch of the second pair of notches has a second predetermined radius, and wherein the first pair of notches are separated by a first predetermined thickness and the second pair of notches are separated by a second predetermined thickness.

9. The system of claim 5, wherein the two compliant portions include a first reduced diameter section having a first predetermined length and a second reduced diameter section having a second predetermined length.

10. The system of claim 1, wherein the at least one compliant portion includes a reduced diameter section having a predetermined length.

11. The system of claim 1, wherein the at least one compliant portion includes a pair of stops configured to limit an amount of flexure of the driver about the at least two axes.

12. A surgical robotic system for driving a bone screw into bone comprising:
a surgical robot including a robot arm and an end effector, the end effector having a plurality of tracking markers and a guide tube;
a compliant orthopedic driver including:
a body extending from a proximal end to a distal end along a driver axis;
a driver tip disposed at the distal end of the body,
wherein the body includes a first compliant portion and a second compliant portion longitudinally spaced apart from the first compliant portion along the body, and
wherein each compliant portion includes:
a first round notch adapted to flex about a first axis transverse to the driver axis; and
a second round notch longitudinally disposed from the first notch and adapted to flex about a second axis transverse to the driver axis, the first axis being angularly offset from the second axis; and
wherein the compliant driver is inserted through the guide tube for driving the bone screw into the bone using the compliant orthopedic driver.

13. The system of claim 12, wherein the first axis is angularly offset from the second axis by 90 degrees.

14. The system of claim 12, wherein the compliant portion includes a pair of oppositely disposed concave notches relative to the driver axis.

15. The system of claim 12, wherein at least one of the compliant portions includes a pair of stops configured to limit an amount of flexure of the body about the at least two axes.

16. The system of claim 12, wherein the first and second compliant portions comprise a first pair of round notch hinges and a second pair of round notch hinges, each of which includes a first pair of round notches and a second pair of round notches angularly offset from the first pair of notches.

17. The system of claim 16, wherein the first pair of notches is perpendicular to the second pair of notches.

18. The system of claim 16, wherein each notch of the first pair of notches has a first predetermined radius and each notch of the second pair of notches has a second predetermined radius, and wherein the first pair of notches are separated by a first predetermined thickness and the second pair of notches are separated by a second predetermined thickness.

19. The system of claim 16, wherein the first and second compliant portions include a first reduced diameter section having a first predetermined length and a second reduced diameter section having a second predetermined length.
